(19) United States
(12) Patent Application Publication
(10) Pub. No.: US 2018/0161091 A9
(43) Pub. Date: Jun. 14, 2018

(54) Radio frequency handpiece for medical treatments

(71) Applicant: Wendy Epstein, Grandview, NY (US)

(72) Inventor: Wendy Epstein, Grandview, NY (US)

(21) Appl. No.: 14/756,848

(22) Filed: Oct. 21, 2015

Related U.S. Application Data
(60) Provisional application No. 62/122,448, filed on Oct. 21, 2014.

(51) Int. Cl. A61B 18/14; A61B 18/12

(52) U.S. Cl. CPC A61B 18/1477; A61B 2018/0047; A61B 2018/143; A61B 18/1206

(57) ABSTRACT

Aspect of the inventions may include a universal handpiece that can be coupled to pre-existing legacy RF signal generator systems to provide certain precision controlled radiofrequency therapies and treatments for skin. Further, another embodiment of the present invention may include an interchangeable electrode (tip section) with an array of distally insulated microneedles, conductive pads or smooth surface configurations which couple to the handpiece for allowing for heating of dermis without damage to the superficial epidermis. This allows the handpiece to be used for various different treatment types by changing the tip or electrode section to meet the requirements of a desired therapy, rather than requiring another dedicated handpiece.

### PRIORITY CLAIM

This application claims priority from U.S. Provisional application Ser. No. 62/122,488 filed Oct. 21, 2014.

### BACKGROUND OF THE INVENTION

There are two biologically distinct aging processes affecting the skin. The first is intrinsic aging, which is essentially caused be the inability of cells to perfectly replicate for an indefinite period of time. The second is extrinsic damage caused by chronic ultraviolet exposure to the sun, and inflammation from acne that can damage or destroy collagen and elastic fibers. This frequently results in a thin dermis with skin laxity, fine lines and wrinkles, and acne scarring, with adverse effects on appearance and personal self esteem.

Various energy based modalities including lasers have been used in the past to improve skin laxity and acne scars. Ablative lasers have significant side effects such as prolonged erythema and post-inflammatory hyperpigmentation, especially in patients with more pigmented skin. Non-ablative lasers are less effective.

Other therapeutic approaches are known as well. For example, fractional radiofrequency micro-needle therapy devices for the treatment of intrinsic and extrinsic damage to the skin are well known in the art.

Many such devices have been U.S. FDA approved and sold for skin tightening (reduction in skin laxity), rejuvenation, improvement of fine lines and wrinkles, and treatment of acne scarring. These devices reduce skin laxity and improve fine lines and wrinkles as a direct result of stimulating the creation of new collagen and elastic fibers within the dermis. The new collagen and elastic fibers add volume and thicken the dermis thus pushing up or elevating indentations (wrinkles and or acne scars) in the overlying skin. These radiofrequency devices have conductive tips that generate alternating current causing friction between water molecules within the dermis and thereby generate heat within the dermis. It is the heat generated in the dermis by these devices that directly cause the desirable clinical effects. Typically, the ideal temperature for dermal heating is about 55-68 degrees Celsius.

CO₂ lasers, in contrast to RF devices, heat up the dermis using light energy rather than high frequency radio wave energy. Radiofrequency energy, on, the other hand, uses the tissue's electrical resistance within the various layers of the skin to transform the applied RF energy into thermal energy. For example, this may be dictated by the following formula: Energy (J)=I²*R*T (where I=current, R=tissue impedance and T=time of application). Because RF energy produces an electrical current instead of a light source, tissue damage can be more focused and precise, and, advantageously, epidermal melanin is minimized. With this knowledge, RF energies can be used for patients of all skin types—that is, it is virtually pigmentation agnostic and allows for different depths of penetration based on what is to be treated, allowing for ultimate collagen contraction and production of new collagen.

In 2002, Thermage was the first to market a RF device for the purposes of skin rejuvenation which was FDA approved in 2009 for non invasive treatment of wrinkles. Since then, various other similar devices have come to market (Exilis, Venus Freeze, Pelleve, Viora Reaction, 3Deep RF, Actent XL, eMatrix, e-Two, TriPolar RF, ReFirme, Sublime, ePrime, Fractora, Evolastin, etc.). The shortcomings of the foregoing include variable results with many patients achieving minimal results, due largely to uncontrolled or limited depth of RF signal delivery.

A more advanced example of one such device is the Infini from Lutronic. This device consists of a radiofrequency source and attached handpiece with an array of disposable insulated microneedles to penetrate the epidermis and thereby deliver interstitial fractionated radiofrequency signals, thus heating only the target tissue, the dermis, leaving the epidermis intact and viable. The pulse duration of the radiofrequency signals range from 100 msec to 1000 msec and is adjustable in 100 msec increments. The depth of penetration of the microneedles is adjustable from 0.5 mm to 3.5 mm in 0.5 mm increments. The power level is adjustable from 2.5W to 50 W in 2.5W increments.

The Infini device is not ideal. The procedure must be repeated over the same surface of skin to allow for several levels of the dermis to be treated by first resetting the needle depth of penetration. This is not ideal as it is not possible to precisely introduce the micro-needles into the exact holes from previous passes, making the treatment inconsistent. The equipment is relatively large in dimension, costly and not interchangeable with any other source of radiofrequency energy.

Several companies manufacture FDA approved light weight, portable, low-powered, affordable radiofrequency devices used for primarily for in office electrosurgery on conscious patients. They are commonly referred to as Hyfrecators. The word hyfrecator is a portmanteau derived from “high-frequency eradicator.” It was introduced as a brand name for a device introduced in 1940 by the Birtcher Corporation of Los Angeles. Today, machines with the name Hyfrecator are sold only by ConMed Corporation. However, the word “hyfrecator” is frequently used as a generalized term to refer to any dedicated non-ground-return electrosurgical apparatus, and a number of manufacturers now produce such machines.

Examples of available hyfrecators include (ConMed Hyfrecator 2000) ConMed Corporation www.conmed.com, Utica, New York ; Aaron 950 Bovie Medical Corporation boviemedical.com; Ellman International, Inc. www.ellman.com and CynoSure (PelleveSF RF generator).

Hyfrecators are commonly used in medicine to destroy tissue, benign or malignant, and for hemostasis during surgery. In operation, hyfrecators emit a low-power (up to 60 Watts) high-frequency high-voltage AC electrical pulses, via an interchangeable electrode (disposable or autoclavable) that is inserted into a removable handpiece that plugs into the hyfrecator. Thus the electrical pulse can be directly introduced to the target tissue to be desiccated, fulgurated or coagulated. The amount of output power is adjustable, and the handpiece device is equipped to accept different tips, electrodes and forceps, depending on the electrosurgical requirement. Standard Hyfrecators are typically set up for monoterminal or biterminal adaptation and to administer monopolar or bipolar current.

Hyfrecators in general are relatively low cost mature technology. However, they are not ideal in many respects and suffer from several drawbacks. First, the electrical pulses are produced in a substantially continuous manner. The ON/OFF and pulse duration of the applied current is determined by the manual operation of the medical practitioner either through depressing the button on the handpiece or depressing of a foot switch. This provides the medical practitioner with limited control over the pulse duration during its application to the patient.

It would be desirable, however, to control the treatment by adjusting or precisely controlling t the duration that the RF signal is applied to the treated area rather than just adjusting power or frequency. It would also be desirable to have automated control over the duration of pulse application. This lack of precision limits the practitioner's ability to provide an optimal therapy. Furthermore, the contact surfaces, or “tips” are limited in size and configuration and those individual tips designed for penetrating into the skin are incapable of being adjusted to provide a controlled depth of penetration. Accordingly, there does not currently exist a universal handpiece capable of connecting to existing legacy RF signal generators that include interchangeable electrode or tip portion with an array of insulated micro-needles or conductive pads suitable for fractional providing radioablation for the purpose of reducing skin laxity and treatment of acne scarring.

Therefore, it would be desirable to provide devices and methods that overcome the shortcomings of such prior art systems.

### SUMMARY OF THE INVENTION

The inventions described herein are unique at least because they include a universal handpiece that may be coupled to pre-existing legacy RF signal generator systems to provide certain precision controlled radiofrequency therapies and treatments for skin. Further, another embodiment of the present invention may include an interchangeable electrode (tip section) with an array of distally insulated microneedles, conductive pads or smooth surface configurations which couple to the handpiece for allowing for heating of dermis without damage to the superficial epidermis. This allows the handpiece to be used for various different treatment types by changing the tip or electrode section to meet the requirements of a desired therapy, rather than requiring another dedicated handpiece.

In embodiments having arrays of microneedles, such microneedles may be adjustable and/or may vary in length will allow for variations in depth of target tissue being treated. Alternatively, variations in insulation length along the shaft of the microneedles may allow for flow of current simultaneously at various depths of the dermis treating the target tissue within the dermis with greater speed and efficiency .,e.g., with one pass). Overall, the technology disclosed herein enables precise and predictable accuracy as to location (depth and volume) of the target tissue in the dermis being treated with a controlled zone of electro-thermal damage . Further, some embodiments may include an epidermis cooling apparatus with the handpiece to prevent surface heat damage during treatment to thereby facilitate providing an optimal and comfortable therapy to the patient. In sum, these inventions will enable greater precision, predictability, efficiency thus overall greater efficacy in producing desired results.

One embodiment of the present invention may include a handpiece for use with a pre-existingsignal generator to provide dermatological treatment having an input section that accepts electrical signals from the pre-existing signal generator, the handpiece further including an output section that couples to a tip section, wherein the tip section configured to provide the electrical signals received by the output section to the skin of a patient; and a control circuitry for selecting a duration that the electrical signals are provided from the input section to the output section.

Another embodiment of the present invention may include a conductive tip section for use with a handpiece that connects to a pre-existing signal generator, wherein the tip section may include an array of conductive surfaces that selectively conduct electrical signals from the pre-existing signal generator to a skin of a patient and a universal input section that couples to the output of the handpiece such that tip sections of a plurality of different conductive surface configurations may be attached to the handpiece.

Another embodiment of the present invention may include a conductive tip section for use with a handpiece that connects to a pre-existing signal generator, wherein the tip section may include a substantially smooth and conductive surface such as those used in the PELLEVE system by CynoSure Corporation or an array of conductive pads such as those used in THERMAGE units by Valeant Pharmacueticals such that the tip section selectively conducts electrical signals from the pre-existing signal generator to the surface of the skin of a patient; and a universal input section that couples to the output of the handpiece such that tip sections of a plurality of different conductive surface configurations may be attached to the handpiece.

### DETAILED DESCRIPTION OF THE INVENTION

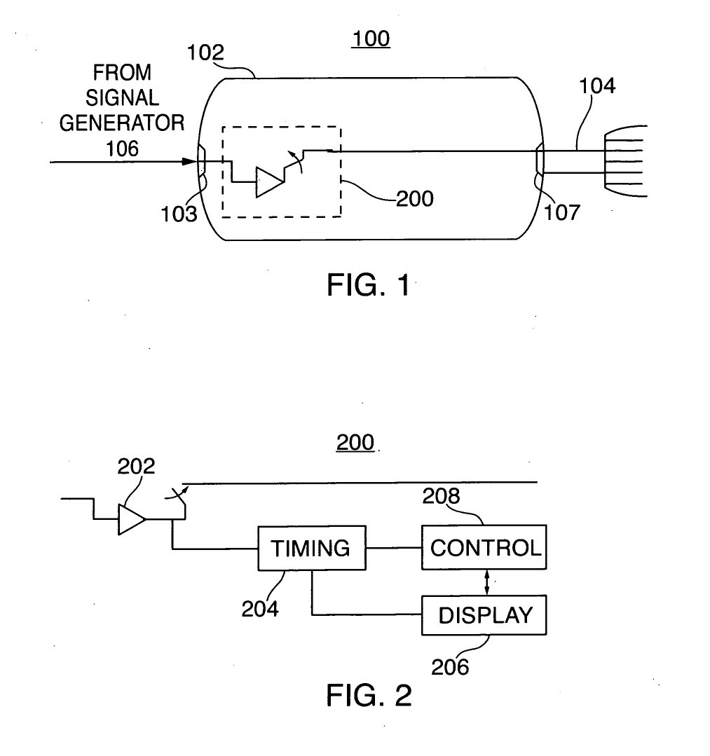

A handpiece 100 constructed in accordance with one embodiment of the present invention is shown in FIG. 1. As shown, handpiece 100 includes wand or handpiece section 102 and tip (or electrode) section 104. In the preferred embodiment, wand section 102 is configured to substantially seamlessly accept the output of a pre-existing desired RF signal generator device 106 (not shown) such as a Hyfrecator through input section 103 (although any pre-existing signal generator may be used if desired.) In some embodiments, it may be preferred the signal generator or “source” is an FDA device approved for use in providing medical treatments.

From a physical perspective, handpiece 102 couples to the output of generator 106 through input section 103 in a known secure way such as by “snap on,” “screw in,” “rotate and lock,” or any other suitable secure physical coupling or connection method known in the art through input section 103. From an electrical perspective, handpiece section 102 and input 103 may be constructed to receive the electrical output of generator 106 such that the received signal is substantially undistorted and looses minimal strength through the, physical connection. This may include impedance and/or material matching or other efficient electrical interconnection techniques known in the art, which allow RF signals to pass through handpiece 102 to tip section 104 substantially unaltered (or altered minimally or in a desired way). One way such connection may be achieved is through cabling configured to connect to the signal output connection on a particular source 106 (e.g., Hyfrecator) and may include impedance matching circuit in input section 103 (not shown) to receive high frequency RF signals (which may be bipolar or monopolar; monoterminal or biterminal in nature).

As shown in FIG. 1, handpiece 102 may also include electronics section 200, including switch 202, timing circuit 204, display 206 (optional) and control input 208. Switch 202 may be any suitable configuration known in the art that periodically turns ON and OFF (e.g., opens and closes) based on certain control signals including solid states switches, relays, transistors, op amps, etc. and the like to pass RF signals in the desired power range. Similarly, timing circuit 204 may be any suitable circuit for periodically commanding switch 202 to turn ON and OFF such as and oscillator, flip flop, pulse generator etc. (e.g., 555, 556 or 558 timer circuits) and may include some programmable memory (not shown) for selecting certain preset functions such as timing options or programming to a desired ON/OFF duration. Control circuit 208 may be any suitable input device such as a physical or virtual button, thumbwheel, touch pad or other tactile device or the like that allows a user to set the timing duration of switch 202 and other control functions further described herein. Lastly, display 206 may be any suitable electronic display known in the art such as LCD or LED display.

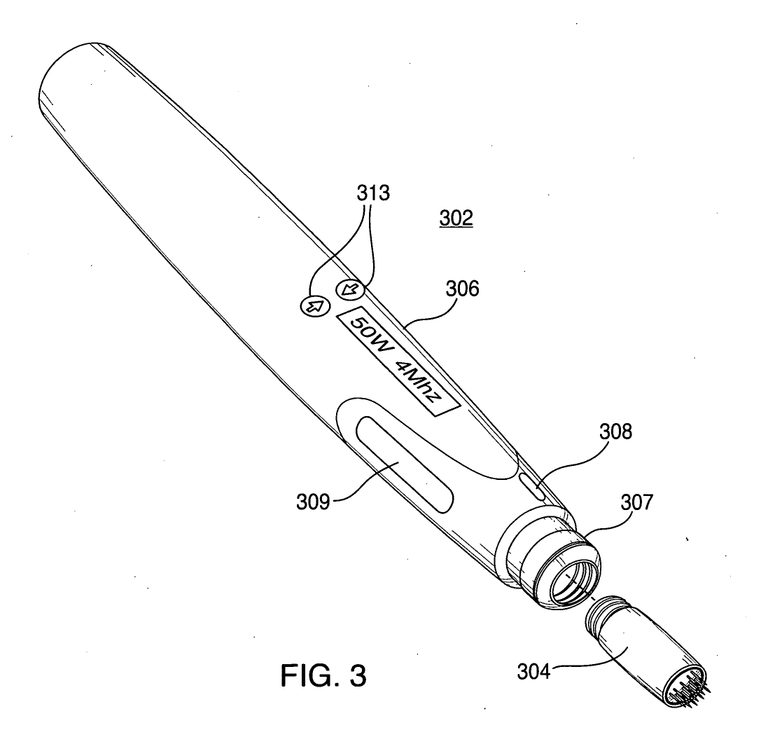

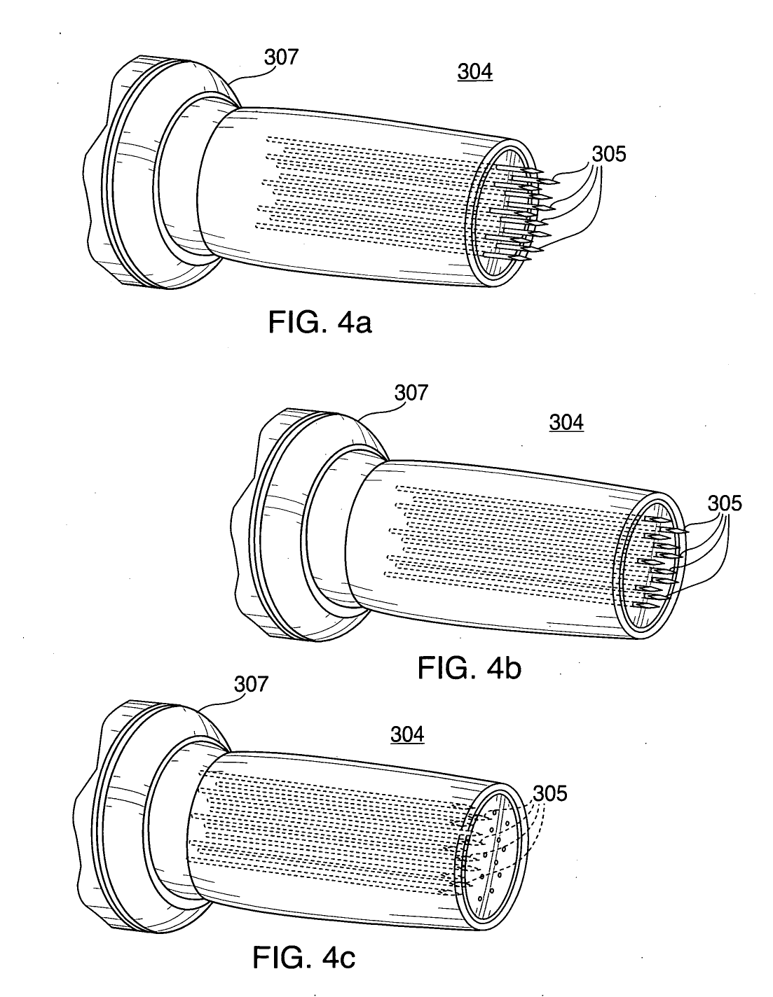

In some embodiments, control circuit 208 may include a “Built in Test” (BIT) feature that measures and displays the frequency and/or power of the RF signal received from the source 106 to ensure the correct signal is being received applied through tip section 104. In certain embodiments, the user may be able to select the power and frequency range of an acceptable input signal to supply through handpiece 102. In the case where the input signal is not within the selected range, handpiece 102 may automatically disable the flow of energy to tip section 104. One way this may occur is the RF signal generator 106 may be set to a particular setting (e.g., 50 watts at 4 Mhz). The user may confirm this is the correct signal by viewing the signal power and magnitude on display 206 (306 in FIG. 3) and accepting this setting by pressing a button on control circuit 208 (313 in FIG. 3) allowing treatment to begin (e.g., energy to flow to tip section 104). Should the signal vary by more than a preset set amount (e.g., 5%). the energy flow to tip section 104 through switch 102 may be disabled. Further, in some embodiments, a user may be able to directly communicate with signal generator 106 through handpiece 102 and program the desired output signal power and/or frequency through control circuit 208 (e.g., through a cable connection back to generator 106 (not shown)).

Further, in some embodiments, handpiece 102 may include an electrical motor (not shown) which reciprocates tip section 104 back and forth. A user, such as a medical clinician, may depress an ON button such as ON button 309 in FIG. 3, which may enable: 1) the electrical motor to begin reciprocating a microneedle array (shown in FIGS. 4), and 2) may allow the electrical signal to flow from source 106 to the tip section. If button 309 is released (or pressed again), treatment may stop. Other embodiments may include a vibration setting that may allow handpiece 102 and tip section 104 to vibrate (e.g., when using a blunt tip or smooth tip section 104 such as the one shown in FIG. 7). Handpiece section 102 may also include one or more LEDs (308 in FIG. 3) coupled to switch 202 or timing circuit 204 that blinks or flashes when switch 202 is conducting or ON (i.e., when the wand is “ON”). In some embodiments, this may also include a slight vibration of handpiece 102 and/or audio sound to indicate switch 202 is conducting (not shown). In this way, an operator can immediately determine whether handpiece 102 is conducting and a signal from generator 106 is being sent to tip section 104 (and ultimately applied to a patient). Further, handpiece 102 may include a sensor that may stop energy flow when it senses loss of contact with a patient's skin such as a pressure sensor in output section 107 (not shown).

In operation, a clinician may desire to select a treatment for a patient. This may include setting the frequency and power (amplitude) of the signal to be sent from a pre-existing signal generator 106 such as a Hyfrecator. This is typically done at the console of the frequency generator (but also may be done at handpiece 102 as described above (not shown)). However, the clinician may also desire to set the duration of the pulse to be provided to tip section 104 with microsecond type precision. In accordance with one aspect of the invention, this may be done using handpiece 102. For example, a user may manipulate control circuit 208 to turn switch 202 ON and OFF at certain selectable intervals (e.g., 100 ms intervals). This means switch 202 would pass the input signal from source 106 to tip 104 every 100 ms (ON) and then interrupt signal flow by opening the connection for the next 100 ms (OFF) and so on. This allows the clinician added control to apply treatment with precision which may allow for a higher strength signal to be applied to the patient for short periods of time, which produces a the desired heating of the dermis.

Handpiece 100 by itself, equipped with a digital pulse duration control is a unique and advantageous. For example, handpiece 100 may be useful not just for fractional radiofrequency treatment of skin laxity, but also for everyday in office surgical procedures allowing for selectable RF microbursts (e.g., about 100 msec-1000 msec) of higher energy for greater control in treating smaller lesions on the skin. This can be done without risk of scarring or collateral damage to surrounding tissue by the selection of shorter pulse durations leading to shorter thermal relaxation times.

The duration of the ON-OFF cycle may be selectable through input device 208 and timing circuit 204 in conjunction with programmable memory (not shown). This may allow the operator to select form a series of preset intervals (e.g., multiples of 100 ms) or program a specific desired ON-OFF cycle (e.g., every 330 ms). Once a duration has been selected, display 206 may ask the user to confirm the displayed interval is correct. They may confirm the setting by and input into control circuit 208. Once confirmed, the user may then issue a command through circuit 208 and/or ON button 309 to initiate the beginning of treatment causing switch 202 to turn ON and OFF as programmed.

Referring now back to FIG. 1, handpiece 100 includes tip section 104. In the preferred embodiment, tip section 104 is configured to substantially seamlessly accept the output of signal generator device 106. From a physical perspective, tip 104 will couple to wand 102 through output section 107 in a known secure way such as by “snap on,” “screw in,” “rotate and lock” or other secure physical coupling or connection method known in the art. From an electrical perspective, tip section 104 may be constructed to receive the electrical output of handpiece 102 through output section 107 such that the received signal is substantially undistorted and looses minimal strength through the physical connection. This may include impedance and/or material matching or other efficient electrical interconnection techniques known in the art, which allow signals to pass from output section 107 to tip section 104 substantially unaltered. In other embodiments, the input signal may be modified in a known way either statically due to materials or interconnection type (i.e., without user interaction) or dynamically (i.e., programmed with user interaction)(not shown).

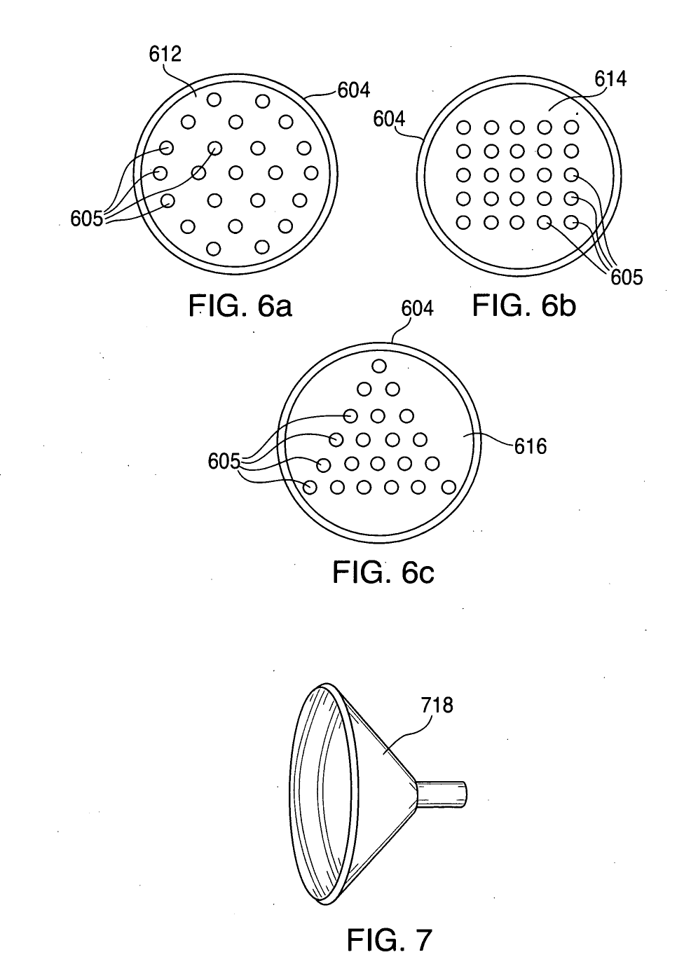

FIG. 3 illustrates one potential embodiment of handpiece 102 as handpiece 302. As shown, handpiece 302 may include some or all of elements described in connection with handpiece 102 and FIG. 1, and may operate in the same or similar way described above . As shown, handpiece 302 may include tip section 304, display 306, output section 307, LED 308, ON Button 309, and control adjustment buttons 313. In operation, user may adjust handpiece 302 settings through buttons 313 and display 306 as further described herein. Depressing ON button 309 may be used to initiate treatment and/or end or interrupt treatment when insufficient pressure is applied. LED 306 may be ON or blink ON and OFF when power is allowed to pass to tip section 304. Output section 307 which may operate and function substantially the same as or similar to output section 107 and couples to tip 304, with the coupling configured such that multiple different tips 304 with multiple different tip configurations such as those shown in FIGS. 6 and 7 may connect to handpiece 304. This allows the handpiece of the present invention to provide multiple types of treatments based on the tip section 304 selected, thus obviating the need for multiple dedicated handpieces with different tip sections 304. Interchangeable tip sections 304 for universal handpiece 302 is one advantageous aspect of the present invention.

FIGS. 4a-4c show how tip section 304 (coupled to output section 307) having an array of microneedles 305 reciprocate back and forth within the body section of tip 304. FIG. 4a shows the microneedles 305 fully extended. In some embodiments, tip section 304 may be configured to conduct power only when the array of needles is fully extended (or within a preset distance of being fully extended). Ili some embodiments, the speed of reciprocation of the array may be selected through control circuit 208 and display 206 (FIG. 2), which in some embodiments may include buttons 313 and display 306 (FIG. 3) (not specifically shown). FIG. 4b shows microneedles 305 as they begins to recede into tip section 304 and FIG. 4c shows array of microneedles fully inside tip section 304. It will be understood that in some embodiments the array may not fully recede into tip section 304 and that FIGS. 4a and or 4b (or 4b and 4c) may represent full reciprocation.

In addition, in some embodiments, the length or degree to which microneedles 305 in the array reciprocate may be selected by adjusting a setting the collar of output section 307 (not shown). For example, microneedles 305 may be 10 mm long. By adjusting a setting on the collar of output section 307, a user may be able to adjust the distance to which the microneedles extend beyond the end of tip section 304 thereby setting a skin puncture depth. Such adjustments may be made in half millimeter increments (or any suitable increment) to allow puncture depths between 0.5 mm and 9 mm although other distances may be used if desired.

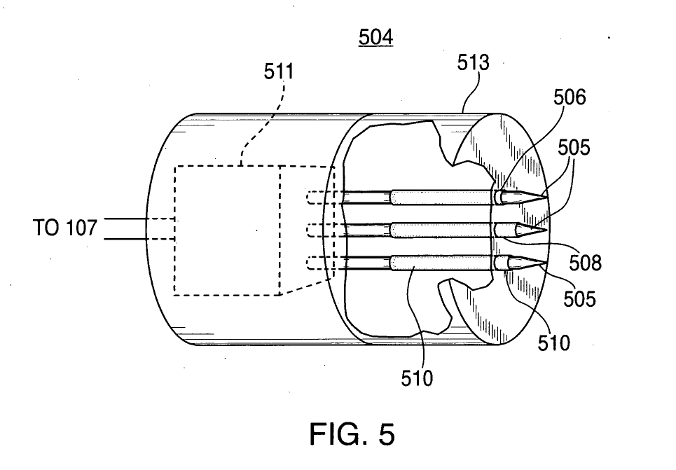

FIG. 5 shows tip section 504 which is a more detailed view of one possible embodiment of tip sections 104 and 304 and may be used with embodiments of handpieces 102 and/or 302. As shown, tip 504 includes microneedles 505 and restrictor sleeve 513. In some embodiments of the invention, micro-needles 505 may be of a fixed length and extend beyond the aperture of restrictor sleeve 513 for a fixed distance. In this case, the degree to which micro-needles 505 may puncture the skin of a patient is determined by this distance (i.e., the distance the needles extended beyond the end of the aperture of sleeve 513 (e.g., 1.5 mm). In other embodiments however, the position of the aperture of restrictor sleeve 513 may be adjustable so the available depth of penetration of microneedles 505 may vary. For example, if microneedles 505 are 5 mm in length, the position of sleeve 513 in relation to needles 505 may be adjusted so the exposed length of needles 505 varies, allowing one tip section 504 to be used for multiple penetration depths, and thus multiple treatment settings. One way this may be accomplished is by spring loading sleeve 513 with certain preset stop points at known distances (e.g., every 0.5 mm) using techniques known in the art and allowing that distance to be adjusted by adjusting a setting on tip 504 or output section 307 (not shown). However, any other suitable approach, such as sliding groove, may be used if desired.

This allows the tip section 504 in the example above to provide microneedles 505 with an effective puncture length from about 0.5 mm-4 mm in 0.5 mm increments. It shall be understood that the foregoing is only illustrative and that other size microneedles and preset distances and needles lengths may be used if desired, and that certain embodiments may not use presets.

In other embodiments, handpiece section 302 may include a motor or other actuation device (not shown) such that tip section 504 (and/or center section 511 and array 505) reciprocates back and forth parallel to the central horizontal axis of handpiece section 302. This may be used to allow micro needles 505 to puncture tissue to a desired depth. For example, the reciprocation distance may be adjustable such that the micro needles puncture to the reciprocation depth. Or, in other embodiments, reciprocation distance remains the same, but the micro needle actuation is controlled by a governor or other limiting mechanism to the desired depth. In either case, the penetration depth may be user adjustable or selectable. Wands with preset penetration depths are also contemplated by the current invention.

In addition, microneedles typically have a certain amount of insulation on the end connected to tip center section 511. This is done to control the amount of conductive surface that this applied to the patients' tissue, which in turn affects how much heat and tissue damage is generated by treatment. This is generally shown by sections 506, 508 and 510, which illustrate some different possible lengths of insulation from tip center section 511. Section 506 represents a microneedle 505 that is covered in insulation up to the beginning of the tip. Section 510 shows less insulation and thus a greater a conductive surface, with section 506 having the least insulation, thereby providing the largest conductive surface. This length may be dependent on various factors such as the materials microneedles 504 are constructed from, the amount of heat desired to be generated and the depth at which treatment is desired and volume of tissue to be treated. It may also depend on micro-needle configuration. Further, the conductivity of the needle material, and the shape, spacing and length of needles in tip 504 may all be adjusted as desired to produce a certain specific desired micro-needle configuration designed to produce or facilitate treatment result.

In some embodiments, tip sections 104, 304, 504 and 604, may have arrays of microneedles 305, 505, 605 wherein individual needles in the array have differing lengths of insulation such that the conductive surface of certain microneedles is greater than that of others. For example, microneedles having greater lengths of insulation may be interspersed randomly or according to a specific pattern within the array of microneedles such that differing depths of dermis are heated during one puncture treatment (not shown). In one such embodiment, microneedles having a greater length of insulation are located substantially at the periphery or center of the microneedle array (not shown). However, any suitable arrangement of varying insulation depths within a microneedle array may be used if desired.

Further, it will be understood that, in some embodiments the microneedles shown herein may be replaced with conductive pads, such that an array or conductive pads is produced, which provides a surface-based skin treatment (not shown). Such arrays may be substantially the same as or similar to the conductive tip sections currently used in conjunction with THERMAGE machines produced by Valeant Pharmaceuticals. Such arrays may, for example, be a substantially square or rectangular array (e.g., 6×6) of conductive pads or sections that operate in a monopolar biterminal fashion that disperse applied RF energy through the skin surface (not shown). Arrays of other sizes (e.g., 3×3, 8×8) or shapes may be used if desired. Such conductive arrays may be created using a sectioned capactive membrane other similar technology.

Referring now to FIG. 6, microneedles 605 may be arranged in any suitable configuration to facilitate treatment. As shown, in circular configuration 612, rectangular configuration 614 , substantially triangular configuration 316, or square configuration (not shown) . Others may also be used if desired depending on treatment or application. The spacing and materials of needles 605 may be selected to produce the desired heating shapes and patterns for a desired volume of tissue.

In some embodiments, a temperature sensor (not shown) may be added to t, tip sections 104, 304, 504 and 604 to make sure the micro needle array is not overheating the treated tissue volume above the desired therapeutic temperature (e.g., about 55-68 degrees Celsius). Some embodiments may further include a cutoff feature that prevents the micro-needle array from heating above a certain temperature. This temperature may be set by a clinician during treatment or by the handpiece manufacturer.

Further, certain embodiments may include a cooling apparatus in the handpeice sections disclosed herein. Such embodiments may include an external source of refrigerant or coolant such as a compressed hydrocarbon that may applied to the treated area sustainably concurrently or somewhat after electrical treatment has been deployed to a certain section of skin. In some embodiments, the coolant may be provided to the skin surface in a computerized, precision controlled, time delayed fashion through a distribution tube attached to the handpiece from the external coolant source (not shown). This provides maximum comfort to the patient as well as providing practitioners with way to provide an optimal or high powered treatment to the patient without damaging skin or causing discomfort, thereby improving the likelihood of a desirable outcome.

Moreover, although the inventions herein have been described in connection with conductive microneedle array or pads, it will be understood that the tip sections may include smooth surface type tips such as those used in conjunction with the PELLEVE system produced by Ellman or CynoSure Corporation. Such a smooth surface type conductor tip is shown as tip section 718 in FIG. 7. Such tips may be constructed in various diameters from 2.5 mm-50 mm depending on intended application. Smaller diameters for around the eyes or mouth, larger diameter for center of cheeks, legs etc. In some embodiments, tip section 718 may be outwardly rounded and come to high point substantially in its center.

In addition, it will be understood that the inventions herein may operate in either monoterminal or biterminal mode. Typically the biterminal mode requires the use of a return in path, commonly in the form of a return plate placed behind the patient during treatment that produces the applied signal with a return path to ground.

Thus, it is seen that a handpiece for use with pre-existing RF signal generators for use in medical treatments is provided. The handpiece may be used with substantially any pre-existing signal generator 106 such as a Hyfrecator, but may include other signal generators produced by Ellman Corporation, Bircther Corporation, CynoSure Corporation or Valeant Pharmacueticals among others. It will be understood that the foregoing is only illustrative of the principles of the invention, and that various modifications can be made by those skilled in the art without departing from the scope and spirit of the invention

Persons skilled in the art will appreciate that the present invention can be practiced by other than the described embodiments, which are presented for purposes of illustration rather than of limitation, and the present invention is limited only by the claims which follow.

What is claimed is:

1. A handpiece for use with a pre-existing signal generator to provide dermatological treatment, comprising:
an input section that accepts electrical signals from the pre-existing signal generator;
an output section that couples to a tip section, the tip section configured to provide the electrical signals received by the output section to the skin of a patient; and
control circuitry for selecting a duration that the electrical signals are provided from the input section to the output section.

2. The handpiece of claim 1 further comprising an electrical motor for actuating the tip section.

3. The handpiece of claim 2 wherein the tip section includes an array of microneedles configured to puncture an epidermal layer of the skin of the patient and provide treatment to a dermal layer.

3. The handpiece of claim 2 wherein the tip section is substantially, blunt and includes an array of conducting pads applied to the outer epidermal layer of patient skin.

4. The handpiece of claim 3 wherein the depth of the epidermal puncture is controlled by an adjustment to the output section of the handpiece.

5. The handpiece of claim 4 wherein the depth of the epidermal puncture is caused by a reciprocation of the array of microneedles.

6. The handpiece of claim 3 wherein the electrical signal is allowed to pass through the output section and to the microneedle array when a puncture depth of the epidermis is substantially at a maximum depth.

7. The handpiece of claim 3 wherein the depth of the epidermal puncture is governed by a restrictor sleeve of the handpiece.

8. The handpiece of claim 1 further comprising a built in test circuitry to test the magnitude or frequency of an output signal produced by the pre-existing signal generator.

9. The handpiece of claim 1 wherein the control circuit includes a plurality of preset signal durations selectable by a user of the handpiece.

10. The handpiece of claim 1 wherein the control circuit is configured to connect to the pre-existing signal generator and to control the output power or frequency of the signal provided to the handpiece.

11. The handpiece of claim 1 wherein the pre-existing signal generator is a Hyfrecator.

12. The handpiece of claim 1 wherein the control circuit includes display circuitry.

13. The handpiece of claim 1, wherein the handpiece includes further temperature sensor circuitry.

14. The handpiece of claim 13 wherein the control circuit is configured to interrupt the transmission of the electrical signal to the tip section if a sensed temperature exceeds a threshold value.

15. The handpiece of claim 1 wherein the control circuit is configured to interrupt the transmission of the electrical signal to the tip section if a insufficient pressure is applied to an ON button.

16. The handpiece of claim 1, wherein the handpiece is configured to include cooling apparatus to cool the epidermal surface layer around a treatment area.

17. The handpiece of claim 1, wherein the output section is configured to connect to tip sections that have differing areas of conductive surfaces.

18. The handpiece of claim 1, wherein the tip section includes an array of microneedles configured to puncture an epidermal layer of the skin of the patient and provide treatment to a dermal layer or is substantially blunt and includes an array of conducting pads applied to the outer epidermal layer of patient skin.

19. The handpiece of claim 1, wherein the tip section is substantially smooth and substantially circular in circumference and the electrical signal is applied to the outer epidermal layer of patient skin.

20. The handpiece of claim 1, wherein the tip section is about 2 mm-35 mm in circumference.

21. A conductive tip section for use with a handpiece that connects to a pre-existing signal generator comprising:
an array of conductive surfaces that selectively conduct electrical signals from the pre-existing signal generator to a skin of a patient;
a universal input section that couples to the output of the handpiece such that tip sections of a plurality of different conductive surface configurations may be attached to the handpiece.

22. The conductive tip section of claim 21 wherein the array of conductive surfaces includes an array of microneedles for puncturing a surface of the patient's skin.

23. The conductive tip section of claim 21 wherein at least a portion of the array of microneedles are insulated.

24. The conductive tip section of claim 23 wherein the array of microneedles are insulated such that only a preselected distal portion of the microneedles can conduct the electrical signal from the pre-existing signal generator.

25. The conductive tip section of claim 21 wherein certain microneedles within the array of microneedles are insulated to different lengths such that the conductive surface of certain microneedles are greater than others.

26. The conductive tip section of claim 21 wherein microneedles insulated to different lengths are interspersed within the array of microneedles.

27. The conductive tip section of claim 16 wherein the array of conductive surfaces includes an array of conductive pads.

28. A conductive tip section for use with a handpiece that connects to a pre-existing signal generator comprising:
a substantially smooth and conductive surface that selectively conducts electrical signals from the pre-existing signal generator to a skin of a patient;
a universal input section that couples to the output of the handpiece such that tip sections of a plurality of different conductive surface configurations may be attached to the handpiece.

29. The conductive tip section of claim 28 wherein the conductive surface is substantially circular or oval in shape.
